(19) United States
(12) Patent Application Publication
(10) Pub. No.: US 2022/0233350 A1
(43) Pub. Date: Jul. 28, 2022

(54) OCULAR MEDICATION DELIVERY APPARATUS

(71) Applicants: Marcus Van Gorden, Wallingford, PA (US); Drew Hofmann, Newburgh, NY (US)

(72) Inventors: Marcus Van Gorden, Wallingford, PA (US); Drew Hofmann, Newburgh, NY (US)

(21) Appl. No.: 17/618,630

(22) PCT Filed: Apr. 14, 2019
(86) PCT No.: PCT/US19/27418

§ 371 Date: Dec. 13, 2021

(51) Int. Cl. A61F 9/00

(52) U.S. Cl. CPC A61F 9/0026

(57) ABSTRACT

An eye medication delivery system and apparatus are disclosed. Preferred embodiments of the inventive system and apparatus provide a gravity vector independent means for safe delivery of a metered mist dosage of ocular medication to a patient's eye and in particular to the cornea. A non-intrusive eye cup is shaped to hold the patient's eye open by a slight pressing action against the orbital socket. This same pressure action activates the metered dosage mist and provides a simple single action for patients to quickly and effectively apply eye medication. In several other embodiments, the apparatus may include a flip up lid to cover and protect the eye cup while not in use and to prevent accidental discharge of the medication. In other embodiments, the apparatus may also incorporate a separate user activation trigger to alternatively control dispensing of the ocular medication.

### FIELD OF THE INVENTION

The present invention generally relates to medication delivery devices, and more specifically relates to ocular medication delivery devices. In one particular embodiment the eye medication delivery system and apparatus uses a gravity-vector-independent delivery mechanism for safe delivery of a metered mist dosage of medication to a patient's eye and in particular to the cornea.

### BACKGROUND DESCRIPTION OF THE FIELD OF THE INVENTION

There are many reasons and times when eye drops or eye medication need to be used by various patients or users. Dry eyes, eye injuries, laser eye surgery, and so on are just some of the reasons that eye medications may need to be applied to or placed on the exterior of the eye or cornea of a user. The standard or traditional way to apply eye medication is by allowing liquid drops to drip or fall onto an open eye from a squeeze dispenser, and thereby flood the exterior of the eye.

This action of having a liquid drop fall, by gravity, onto the cornea is fraught with problems. If the eye dropper is not located above the eye, the drop falls onto the patient's cheek or nose, or forehead. If the patient is not holding his or her eye open, and if the drop is not timed appropriately, the drop may simply fall onto the patient's closed eye. Moreover, even if the patient can try to hold his or her eye open with one hand, and hold the medication container with his or her other hand, by the time the drop releases from the container tip, the patient may blink and the medication may only partially be administered. Accordingly, more often than not, the application of the drops does not make it into the eye at all. The patient either blinks, thereby blocking the medication from getting to the eye, or the patient just misses in placing the drops where needed.

Even if the user has the dexterity to position the eye medication device over his or her eye, and is able to hold his or her eye open with his or her other hand, and is able to time the release of the medication onto his or her cornea, and the user does not inadvertently blink, then almost always the result is that the user's eye reacts by tearing to wash the liquid or medication out. In each of the former scenarios, the medication is simply wasted, lost, or substantially diluted. Due to mis-administered medication, many patients run out of eye medication early, and may not complete their full course of treatment, which often times results in decreased prescription effectiveness.

In the dilution scenario, which is known and expected by physicians and pharmaceutical manufacturers as a natural reaction to tear up and attempt to flush the foreign medication out of the eye. Because of this known effect and reaction, physicians accordingly either compensate by increasing the concentration of the medication within the fluid, or increasing the medication dosage (number of drops). In either scenario (over prescribing medication, or increasing concentration), there is a substantial inefficient use of medication and increased cost.

Additionally, there is also plenty of room for patients to make an error and touch the tip of the eye drop applicator to their eye in order to ensure they deliver the medication to the eye. This can lead to the tip being unsanitary and cause and infection, or the patient can simply injure their eye by scratching the cornea with the applicator tip. Either result is very problematic for the user.

Several devices and systems for eye medication delivery have been designed and manufactured for years. Examples of such devices and systems include U.S. Pat. Nos. 7,524,511 and 8,936,021. While these devices relate to administering an aerosolized spray of medication to one's eye, neither provides any means to ensure that the medication will make it to the eye surface because there is no means for holding one's eye open while the medication is being released. Other patents disclose certain devices and systems that have particular uses and appear to address certain issues, but none provides a comprehensive solution for the overall objective of being able to effectively and safely administer eye medication by an orientation (gravity) independent delivery system using a metered mist dosage.

Accordingly, while certain of designs, devices, and apparatus have been developed and commercialized to address some of the noted issues relating to the application and use of eye medications, none have fully addressed the noted issues and problems. What is needed is an eye medical delivery device and system that provides an orientation independent means for delivery of a metered mist dosage of ocular medication to a patient's cornea that safe to use. Such a device and system has not been created, disclosed, or used in the prior art.

### SUMMARY OF THE INVENTION

The present invention overcomes the disadvantages of the known prior art and fulfills the needs described above by providing an eye medication delivery system and apparatus using a gravity-vector-independent safe delivery means to provide a metered mist dosage of ocular medication to a patient's eye and specifically to the patient's cornea.

A preferred aspect of the invention is an ocular medication delivery apparatus comprising (a) a main body having an internal cavity shaped and sized to hold an ocular medication container; (b) an ocular orbit shaped cup element having a proximal end shaped and sized to fit within said main body cavity, and having a distal end sized and contoured to comfortably fit around a patient's ocular orbital socket and thereby hold said patient's eye open when said ocular orbit shaped cup element is pressed against said patient's ocular orbital socket; and (c) an ocular medication container that fits within said main body internal cavity, wherein when said ocular orbit shaped cup element proximal end is pressed against said ocular medication container, a metered dosage of medication is released as a mist onto said patient's eye and cornea.

A further preferred embodiment of the invention is a method of administering ocular medication using an ocular medication delivery apparatus providing an aerosolized metered mist dosage of said eye medication, wherein said delivery apparatus comprises (a) a main body having an internal cavity shaped and sized to hold an aerosolized medication container; (b) an eye orbit shaped cup element having a proximal end shaped and sized to partially fit within said main body cavity, and having a distal end sized and contoured to comfortably fit around a patient's eye orbital socket and thereby hold said patient's eye open when said eye orbit shaped cup element is pressed against said patient's eye orbital socket; and (c) an aerosolized medication container that fits within said main body internal cavity, said method comprising the steps of (1) engaging said eye orbit shaped cup element within said main body such that in said engaged position said eye orbit shaped cup element may be pressed against said aerosolized medication container; and (2) pressing said eye orbit shaped cup element distal end against a user's eye socket such that said eye orbit shaped cup element proximal end engages said aerosolized medication container, wherein with said engaging of said aerosolized medication container, a metered dosage of medication is released as a mist onto said patient's eye and cornea.

### BRIEF DESCRIPTION OF THE DRAWINGS OF CERTAIN EMBODIMENTS

To easily identify the discussion of any particular element or act, the most significant digit or digits in a reference number refer to the figure number in which that element is first introduced.

FIG. 1 is a perspective view of an embodiment of the ocular medication delivery device in its inert state showing the device handle and the eye cup.

FIG. 2 is a front view of an embodiment of the ocular medication delivery device in its inert state showing the device handle and eye cup.

FIG. 3 is a side view of an embodiment of the ocular medication delivery device in its inert state showing the device handle and eye cup.

FIG. 4 is a top view of an embodiment of the ocular medication delivery device in its inert state showing the device handle and eye cup.

FIG. 5 is perspective view of an embodiment of the ocular medication delivery device showing the device main body.

FIG. 6 is a side view of an embodiment of the ocular medication delivery device showing the device main body.

FIG. 7 is an exploded front view of an embodiment of the ocular medication delivery device in its ready state showing the device handle, eye cup, and medication container.

FIG. 8 is an exploded perspective view of an embodiment of the ocular medication delivery device in its ready state showing the device handle, eye cup, and medication container.

FIG. 9 is an exploded side view of an embodiment of the ocular medication delivery device in its ready state showing the device handle, eye cup, and medication container.

FIG. 10 is a perspective view of an embodiment of the eye cup element shown in a cross-sectional view and showing the expansion chamber.

FIG. 11 is a front cross-sectional view of an embodiment of the eye cup element showing the internal channels and orifices.

FIG. 12 is a side view of an embodiment of the eye cup element showing the cross-sectional line for the views shown in FIGS. 10 and 11.

FIG. 13 is a perspective view of an embodiment of the eye cup element of the present inventive apparatus.

FIG. 14 is a top view of an embodiment of the eye cup element of the present inventive apparatus.

FIG. 15 is a perspective exploded view of another embodiment of the present inventive apparatus.

FIG. 16 is a front exploded view of another embodiment of the present inventive apparatus.

FIG. 17 is a side exploded view of another embodiment of the present inventive apparatus.

FIG. 18 is a perspective front and back view of an embodiment of the present inventive apparatus with a fully enclosed circumference for the eye cup element.

FIG. 19 is a perspective front and back view of another embodiment of the present inventive apparatus with a partially open circumference for the eye cup element.

FIG. 20A is a perspective rendering of an embodiment of the ocular medication delivery device showing it in a stored or inert configuration.

FIG. 20B is a perspective rendering of an embodiment of the ocular medication delivery device showing it converting from its inert state to its ready state.

FIG. 20C is a further perspective rendering of an embodiment of the ocular medication delivery device showing it converting from its inert state to its ready state.

FIG. 21 is rendering showing use of an embodiment of the ocular medication delivery device with a human patient.

### DETAILED DESCRIPTION OF CERTAIN EMBODIMENTS

An innovative eye medication delivery device and system is described in detail in the following paragraphs, including a description of the several elements of the device and system, and the several uses and modes of operation of the device and system.

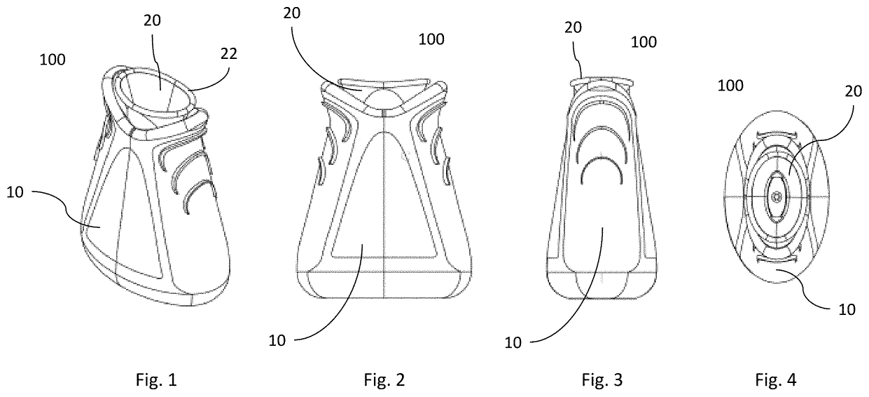

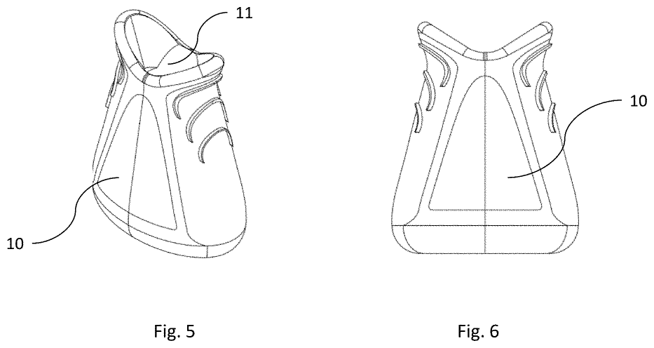

Illustrated in FIG. 1 is a perspective view of the device 100 shown in a stored, closed, or inert configuration. In this embodiment, the device 100 has two primary elements. A first base element, or main body 10 having an oblong shape that provides certain ergonomic features for improving ease of use of the device 100. The main body 10 has an internal cavity 11 that is shaped and sized to accept at least two items. The first item or element is an eye orbit shaped cup element 20, shown in FIG. 1 in the main body 10, and also shown in FIGS. 5 and 6. The second item or element is a medication container 30, as described in further detail below.

As shown in FIG. 1, only the distal or top end of the eye orbit shaped cup element 20 is shown. A front view of the device 100 is further shown in FIG. 2, and further shows additional detail of the shape of the distal end of the cup element 20. More particularly, the cup element 20 has a distal end shape 21 that is oblong or oval so as to encircle a patient's eye, and to fit around the patient's eye and within or fitting on the patient's orbit bone structure. The cup element distal end shape 21 may also have a softer or flexible lip covering 22 to provide a level of comfort when the cup element distal end shape 21 is place around the patient's eye and against the patient's eye orbital socket. A further view of the device 100 is shown in FIG. 3 from a side perspective, and from a top view as shown in FIG. 4.

Additional views of the main body 10 of the device 100 are further shown in FIGS. 5 and 6. As shown in one embodiment, the main body 10 incorporates several ergonomic features including over-molded grips 15 on both side sections of the main body 10. These over-molded grips 15 provide sensory feedback and assistance to the patient to properly hold and operate the device 100, while also providing assistance to the patient to have a firm grip of the device 100 during operation. A side view of the device 100, shown in Fig.6, provides another view of the over-molded grips 15 that may be integrally formed on the exterior of the main body 10.

The internal cavity 11 is shown in FIG. 5 from a top perspective view of the main body 10. In this embodiment, the internal cavity 11 is positioned centered within both X and Y axes of the main body 10 when viewed from above. In another embodiment of the main body 10, the internal cavity 11 may incorporate a ledge section that could provide a physical limit to the motion of the cup element 20 into the internal cavity.

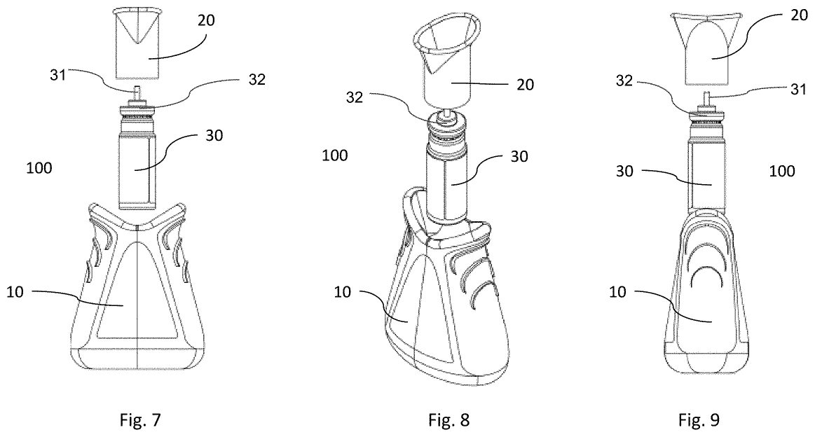

The second item or element that fits within the main body 10 is a medication container 30. FIGS. 7 through 9 show, respectively, a front, perspective, and side blown up views of how the three elements (the main body 10, the cup element 20, and the medication container 30) fit within each other. More particularly, FIGS. 7 through 9 present blown-up views with the medication container 30 positioned above the internal cavity 11 formed within the main body 10, and showing the cup element 20 positioned above the medication container 30. In a fully assembled and ready configuration, the medication container 30 fits snuggly within the internal cavity 11 and the cup element 20 similarly fits within the internal cavity 11 and rests upon the dispensing nozzle 31 of the medication container 30. In operation, when the cup element 20 is slightly pressed towards or into the main body 10, further into the internal cavity 11, and engages the dispensing nozzle 31 of the medication container 30, a valve 32 within the medication container 30 is opened, and a metered dosage of the eye medication is dispensed through a misting valve 22 in the cup element 20.

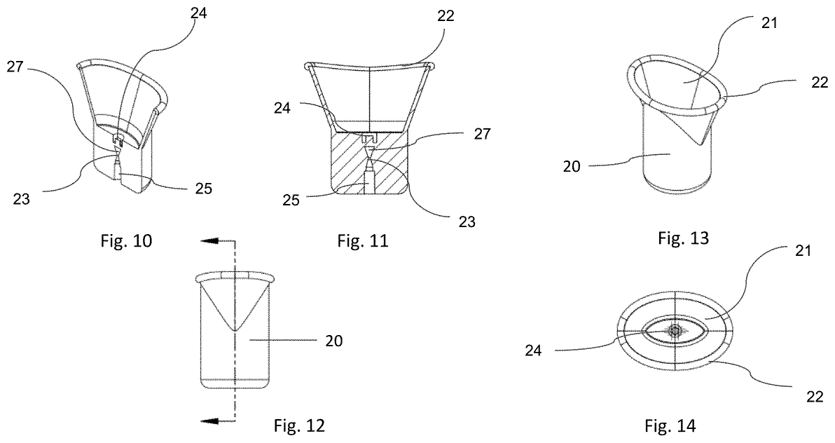

An embodiment of the pressure reduction channel 23 formed within the eye cup element 20, as shown in FIGS. 10 and 11, is designed to reduce the pressure of the medication before the medication is released to and through the atomizer 24. More particularly, FIGS. 10 and 11 show two cutaway views of the cup element, and detailing an embodiment of the pressure reduction channel or valve 23 the cup element 20. As shown, the pressure reduction channel 23 and atomizer 24 ensure that the medication released into the cup el ement 20 and onto the patient's eye is at a very low pressure, very low speed, and results in a gentle uniform application of the medication the patient's eye.

As described, the cup element 20 has an orifice or cavity 25 into which the dispensing nozzle 31 fits when the cup element 20 is placed on top of the medication container 30. The cavity 25 shown in FIGS. 10 and 11 fits over the dispensing nozzle 31 such that when the cup element 20 is pressed slightly into the main body 10 and towards the medication container 30 (held within the main body internal cavity 11), the dispensing nozzle 31 is depressed slightly into the medication container 30 opening medication container valve 32. With the opening of the medication container valve 32, a metered dosage of the eye medication is released through the dispensing nozzle 31 and is expelled through the pressure reduction channel 23 and atomizer 24 shown in FIGS. 10 and 11.

The design of the pressure reduction channel 23 and atomizer 24 is to ensure a low pressure, low velocity mist of the metered dosage of the eye medication is released into the wide cup element distal volume 27. As shown, the cup element internal cavity is designed with a larger expansion chamber 27 ensuring there is a reduction in the pressure of the medication that is released from the medication or aerosol canister 30. This reduction in pressure of the dispensed medication before and after the dosage is expelled through the atomizer further reduces the medication velocity and accordingly allows the device to be used in close proximity to the eye, and provide a gentle misting of the medication to the user's eye.

In further detail, FIGS. 12 through 14 show additional details of an embodiment of the cup element 20. More particularly, FIGS. 12 through 14 show, respectively a side, perspective, and top view of the cup element 20 in isolation. In this embodiment, the shape of the cup element 20 with the oval distal end fits around or contoured to fit within and engage with the patient's eye or orbital socket, while the proximal end has more of a circular or cylinder cross-section to fit within the main body internal cavity 11.

Also, shown in FIGS. 10 through 14 is an example of a softer over molded lip 22 on the cup element 20 that provides a comfortable surface that is in contact with the patient's orbital socket. The FIG. 13 view, being a top view of the cup element 20 shows a top view of the atomizer component 24 of the cup element 20. The section of the cup element 20 onto which the atomizer 24 fits is shown in the two cutaway views of FIGS. 10 and 11. This small element 24 would fit into the eye cup element 20 to provide consistent, uniform, and accurate full atomizing of the metered dosage of medication so as to create a comfortable and gentle mist that can evenly coat the patient's eye. An advantage of having the atomizer 24 not being formed integrally within the eye cup element 20 is that different types of atomizers 24 and orifices formed within the atomizers may be used to create different dispersion patterns for the metered dosage or simply for different types of medication, including different medication viscosities. FIGS. 10 and 14 also show views of the eye cup element 20, illustrating the tapered bottom or proximal end of the cup element 20 that engages with the medication container 30 when the cup element 20 is placed within the main body internal cavity 11.

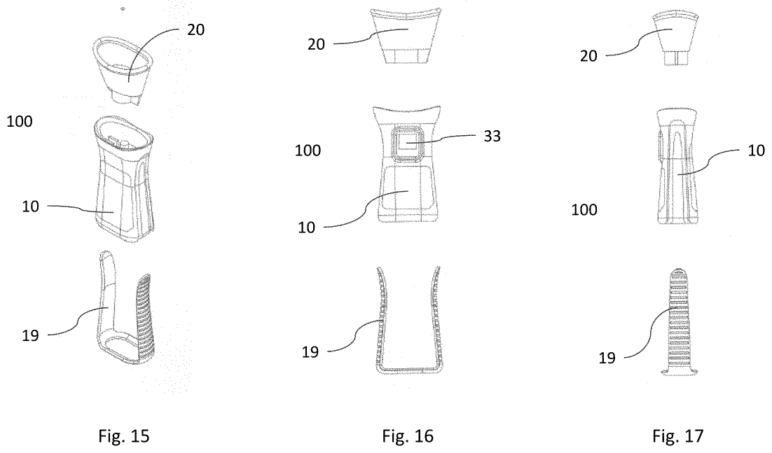

Two alternative embodiments and designs for the ocular medication delivery device 100 are shown in FIGS. 15 through 19. FIGS. 15 through 17 show perspective, front, and side exploded views of an alternative embodiment of the medication delivery device having three core elements. Those elements are the cup element 20, the main body 10, and then a side element or side band 19 that runs along both sides of the main body 10 and along the bottom of the main body.

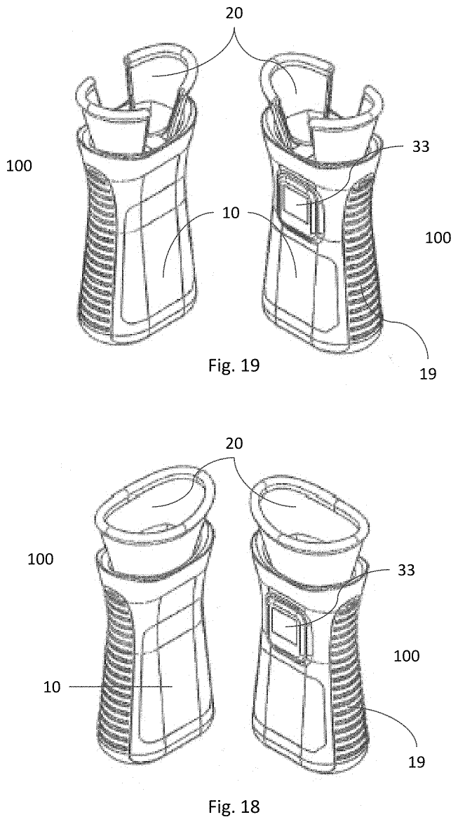

A further alternative embodiment of the ocular medication delivery device 100 is shown in FIGS. 18 and 19 where a full circumference cup element 20 is shown in FIG. 18, and a partially open cup element 20 is shown in FIG. 19. The partially open cup element 20 allows for outside air to intermix with the medication being dispensed from the device 100. Moreover, the partially open cup element 20 ensures that the pressure within the cup element 20 when the mediation is dispensed is essentially maintained at the surrounding atmospheric pressure.

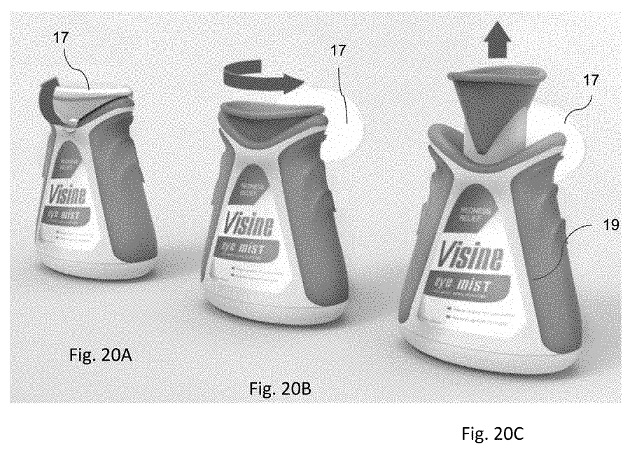

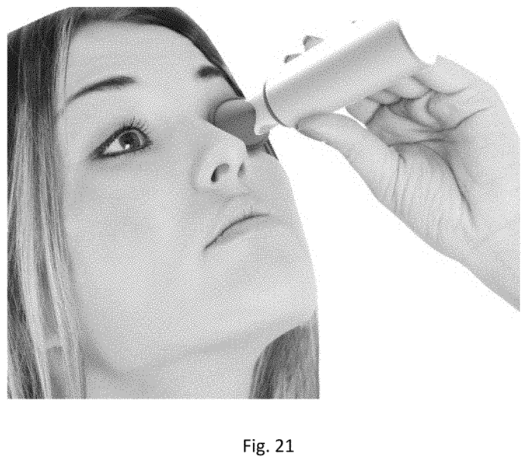

In a further embodiment of the medication delivery device 100, FIGS. 20A through 20C show a perspective rendering of the device with a pivotable lid 17 affixed to the main body 10. The three views shown present how a user may operate the device 100. More particularly, the left most image of FIG. 20A shows the device 100 fully closed and inert with the lid 17 closed. In the middle image of FIG. 20B, the lid 17 is lifted or pivoted upwards. With the lid 17 fully open, the cup element 20 is accessible and may be lifted upwards and rotated (as shown in FIG. 20C, the rotation is approximately 90 degrees). With the cup element 20 rotated and in position and active for medication, illustrated in FIG. 20C, the user need only slightly and gently press the cup element 20 against his or her orbital socket to engage the cup element 20 against the medication container 30, which then opens the medication container 31 valve, resulting in a gentle misting or aerosolizing of the proper metered dosage eye medication coating the user's eye.

Important in this operation is the engaging of the cup element 20 against the user's orbital socket. With the slight pressure of the cup element 20 against the user's orbital socket, the device 100 maintains the user's eye effectively open, and with the same pressure activating the release of the metered dosage from the medication container 30, the user easily and comfortably has the proper and correct eye medication administered.

In another embodiment also having a pivotable lid 17 attached to the main body 10, the cup element 20 may be partially spring loaded such that when the pivotable lid 17 is pivoted the cup element is urged upwards and automatically rotated such that the medication delivery device 100 is fully engaged and ready for usage upon the opening and pivoting of the lid 17 into an open configuration.

FIG. 20A shows the device from a front perspective view, and shows the eye cup component fully stored within the device main body, and with the lid fully engaged and closed, making it easy to store the device 100 in bag or drawer without fear of it accidentally dispensing medication. The over molded soft touch components are also shown in FIGS. 20A through 20C on the outside of the device along the sides of the main body 10. These components could be manufactured as a harder thermoplastic or as soft touch over molded components.

In another embodiment of the delivery device 100, an additional or alternative release mechanism may be incorporated into the main body 10 and device 100. More particularly, as illustrated in FIG. 20C, one or both of the side elements 19 may be configured and engageable such that with a slight compression force to one or both of the side elements, the dispensing nozzle of the medication container 30 may be depressed to release the metered dosage into the eye cup element 20. Such a configuration may be useful for the administration of eye medication to a patient needing assistance, including for example a child, or an older individual, or even for non-human patients such as dogs or horses. In another configuration, a separate release mechanism may be a push button 33 located on the bottom or side of the main body 10, or a push button 33 formed into one or both of the side elements 19.

Along those lines, the delivery device 100, in particular the eye cup element 20, may be configured and sized for different members of the population, including those with smaller facial characteristics (e.g., females) or larger facial structure (e.g., males). Similarly, the delivery device 100 may be configured, sized, and shaped for use with non-human patients, including as noted above, dogs, cats, horses, or any other animal that may benefit from having eye medication administered gently and effectively. An example of use of the device 100 is shown in FIG. 21 with a female administering the eye medication.

While several preferred embodiments and features of the inventive ocular medication delivery device 100 have been described and disclosed, in particular with reference to certain figures and drawings showing certain exemplary embodiments that relate to various particular sized and shaped apparatus, such devices and the disclosed designs as shown are not to be construed as limiting the scope of the inventive device or inventive products. For example, as described above, the medication delivery device 100 is shown for use with human patients. The device has equal utility and application, in potentially different sizes, for canines, felines, equines, or other animals. While described herein with the eye cup element and medication container being separate elements, such elements could be combined for ease of manufacturing and use purposes. All such alternate embodiments are believed to be within the scope of the inventive design and the below claims.

It will be recognized by those skilled in the art that other modifications, substitutions, and/or other applications are possible and all such modifications, substitutions and applications are within the true scope and spirit of the present invention. It is likewise understood that the above disclosure and attached claims are intended to cover all such modifications, substitutions, and/or applications.

What is claimed as the invention is:

1. An ocular medication delivery apparatus, comprising:
a main body having an internal cavity shaped and sized to hold an ocular medication container;
an ocular orbit shaped cup element having a proximal end shaped and sized to fit within said main body cavity, and having a distal end sized and contoured to comfortably fit around a patient's ocular orbital socket and thereby hold said patient's eye open when said ocular orbit shaped cup element is pressed against said patient's ocular orbital socket; and
an ocular medication container that fits within said main body internal cavity;
wherein when said ocular orbit shaped cup element proximal end is pressed against said ocular medication container, a metered dosage of medication is released as a mist onto said patient's eye and cornea.

2. The ocular medication delivery apparatus of claim 1, wherein said main body and ocular orbit shaped cup element are over molded using at least one of a thermoplastic and thermoset material.

3. The ocular medication delivery apparatus of claim 1, further comprising flip top lid to cover said ocular orbit shaped cup element distal end.

4. The ocular medication delivery apparatus of claim 3, wherein when said flip top lid is opened, said ocular orbit shaped cup element is rotated into position such that said ocular orbit shaped cup element distal end may be placed against said patient's eye orbital socket, and said ocular orbit shaped cup element proximal end is positioned to actuate said ocular medication container.

5. The ocular medication delivery apparatus of claim 1, further comprising a separate actuating mechanism on said main body for a medical professional to actuate said ocular medication container.

6. The ocular medication delivery apparatus of claim 1, wherein said ocular medication container contains medication in an aerosol state.

7. The ocular medication delivery apparatus of claim 1, wherein said ocular orbit shaped cup element is sized and shaped for use by females within the range of 10^{th} to 90^{th} percentile eye orbit size.

8. The ocular medication delivery apparatus of claim 1, wherein said ocular orbit shaped cup element is sized and shaped for use by males within the range of 10^{th} to 90^{th} percentile eye orbit size.

9. The ocular medication delivery apparatus of claim 1, wherein said ocular orbit shaped cup element is sized and shaped for equine use.

10. The ocular medication delivery apparatus of claim 1, wherein said ocular orbit shaped cup element is sized and shaped for canine use.

11. The ocular medication delivery apparatus of claim 1, wherein said ocular orbit shaped cup element is sized and shaped for feline use.

12. A method of administering ocular medication using an ocular medication delivery apparatus providing an aerosolized metered mist dosage of said eye medication, wherein said delivery apparatus comprises (a) a main body having an internal cavity shaped and sized to hold an aerosolized medication container; (b) an eye orbit shaped cup element having a proximal end shaped and sized to partially fit within said main body cavity, and having a distal end sized and contoured to comfortably fit around a patient's eye orbital socket and thereby hold said patient's eye open when said eye orbit shaped cup element is pressed against said patient's eye orbital socket; and (c) an aerosolized medication container that fits within said main body internal cavity, said method comprising the steps of:
(1) engaging said eye orbit shaped cup element within said main body such that in said engaged position said eye orbit shaped cup element may be pressed against said aerosolized medication container; and
(2) pressing said eye orbit shaped cup element distal end against a user's eye socket such that said eye orbit shaped cup element proximal end engages said aerosolized medication container, wherein with said engaging of said aerosolized medication container, a metered dosage of medication is released as a mist onto said patient's eye and cornea.

13. The method of administering ocular medication using an ocular medication delivery apparatus as described in claim 12, wherein said apparatus further comprises a separate actuating mechanism on said main body for a medical professional to actuate said aerosolized medication container.

14. The method of administering ocular medication using an ocular medication delivery apparatus as described in claim 12, wherein said eye orbit shaped cup element is sized and shaped for use by females within the range of 10^{th} to 90^{th} percentile eye orbit size.

15. The method of administering ocular medication using an ocular medication delivery apparatus as described in claim 12, wherein said eye orbit shaped cup element is sized and shaped for use by males within the range of 10^{th} to 90^{th} percentile eye orbit size.

16. The method of administering ocular medication using an ocular medication delivery apparatus as described in claim 12, wherein said eye orbit shaped cup element is sized and shaped for equine use.

17. The method of administering ocular medication using an ocular medication delivery apparatus as described in claim 12, wherein said eye orbit shaped cup element is sized and shaped for canine use.

18. The method of administering ocular medication using an ocular medication delivery apparatus as described in claim 12, wherein said eye orbit shaped cup element is sized and shaped for feline use.
